(19) United States
(12) Patent Application Publication
(10) Pub. No.: US 2014/0288115 A1
(43) Pub. Date: Sep. 25, 2014

(54) COMPOSITIONS AND METHODS FOR TREATING BRAIN CANCER

(71) Applicants: Thomas C. Chen, Los Angeles, CA (US); Florence M. Hofman, Los Angeles, CA (US); Stan G. Louie, Los Angeles, CA (US); Axel H. Schonthal, Los Angeles, CA (US); Nicos A. Petasis, Los Angeles, CA (US)

(72) Inventors: Thomas C. Chen, Los Angeles, CA (US); Florence M. Hofman, Los Angeles, CA (US); Stan G. Louie, Los Angeles, CA (US); Axel H. Schonthal, Los Angeles, CA (US); Nicos A. Petasis, Los Angeles, CA (US)

(73) Assignee: UNIVERSITY OF SOUTHERN CALIFORNIA, Los Angeles, CA (US)

(21) Appl. No.: 14/298,697

(22) Filed: Jun. 6, 2014

Related U.S. Application Data
(63) Continuation of application No. PCT/US2012/068428, filed on Dec. 7, 2012.
(60) Provisional application No. 61/569,101, filed on Dec. 9, 2011.

(51) Int. Cl. A61K 31/4741; A61N 5/10; C12Q 1/68; A61K 31/495; A61K 45/06

(52) U.S. Cl. CPC A61K 31/4741; A61K 31/495; A61K 45/06; C12Q 1/68; A61N 5/10; C12Q 2600/136
USPC 514/291; 514/393; 435/375; 435/6.18; 600/1

(57) ABSTRACT

It is discovered that noscapine is effective in treating temozolomide (TMZ)-resistant brain cancer. Provided are compositions and methods of treating brain cancer patients, in particular those that are TMZ-resistant. The patients are treated by administration of a therapeutically effective amount of noscapine or an analog thereof. In certain aspects, TMZ is also administered to the patients. Examples of brain cancers include glioma such as glioblastoma multiforme.

### CROSS-REFERENCE TO RELATED APPLICATIONS

This application is a continuation under 35 U.S.C. §120 of International Application No. PCT/US2012/068428, filed Dec. 7, 2012, which in turn claims priority under 35 U.S.C. §119(e) to U.S. Provisional Application No. 61/569,101, filed Dec. 9, 2011, the content of each of which is incorporated herein by reference in their entireties.

### BACKGROUND

Primary brain tumors consist of a diverse group of neoplasms, derived from various different cell lineages. Pursuant to a World Health Organization categorization, tumors of the central nervous system are classified as astrocytic, oligodendroglial, or mixed (oligoastrocytic). These tumors are further classified by subtypes and are graded, based on histology, from I to IV, with grade IV being the most aggressive. Every year, 18,500 new brain tumors are diagnosed in the United States. Of these tumors, 50% are gliomas; 50% of these gliomas are glioblastoma multiforme (GBM), with the dismal survival prognosis of 10-12 months.

The standard of care for glioma treatment is resection, radiation and chemotherapy consisting primarily of temozolomide (TMZ). Patients routinely become resistant to this drug and few options are then available to them.

### SUMMARY

Provided herein is a method of treating a patient suffering from temozolomide (TMZ)-resistant brain cancer, by administering to the patient an effective amount of noscapine or an analog thereof, and thereby treating the patient. In a further aspect, the disclosure provides a method of inhibiting the growth of a brain cancer cell that is resistant to a temozolomide (TMZ) treatment, by contacting the brain cancer cell with an effective amount of noscapine or an analog thereof. In a yet further aspect, each of the above noted methods can be combined with a treatment comprising, or alternatively consisting essentially of, or yet further consisting of, the administration of an effective amount of noscapine or an analog thereof. In one aspect, the method further comprises identifying a patient as having a TMZ-resistant brain cancer, and then after identifying the cancer as such, treating the patient in accordance with the disclosed methods. Although in most part this disclosure is directed to a brain tumor or cell that is resistant to TMZ, the methods, compositions, kits, formulations as disclosed herein are intended to be applicable to the treatment of a central nervous system (CNS) tumor cancer patient (gliomas, menengiomas, pituitary adenomas), a CNS cancer cell metastasis from a systemic cancer, lung cancer cell, prostate cancer cell, breast cancer cell, hematopoietic cancer cell, ovarian cancer cell or brain cancer patient, whose tumor is resistant to temozolomide (TMZ). Compositions and formulations for use in the above noted methods are further disclosed herein.

Thus, one embodiment of the present disclosure provides a method of treating a temozolomide (TMZ)-resistant brain cancer patient, comprising, or alternatively consisting essentially of, or yet further consisting of, administering to the patient an effective amount of noscapine or an analog thereof. In one aspect, the brain cancer is glioma. In another aspect, the glioma is glioblastoma multiforme.

In some aspects, the method further comprises, or alternatively consists essentially of, or yet further consists of, administering to the patient an effective amount of TMZ or an analog thereof. In some aspects, the method further comprises, or alternatively consists essentially of, or yet further consists of, administering to the patient a chemotherapy or radiotherapy.

A particular embodiment of the present disclosure provides a method of treating a temozolomide (TMZ)-resistant glioma patient, comprising, or alternatively consisting essentially of, or yet further consisting of, administering to the patient an effective amount of noscapine and a therapeutically effective amount of TMZ.

Also provided is a method of inhibiting the growth of a brain cancer cell that is resistant to a temozolomide (TMZ) treatment, comprising, or alternatively consisting of, or yet further consisting of, contacting the brain cancer cell with an effective amount of noscapine or an analog thereof. In one aspect, the contacting is in vivo, or alternatively in vitro.

Still provided, in one embodiment, is a method of treating a brain cancer patient, comprising, or alternatively consisting essentially of, or yet further consisting of, administering to the patient an effective amount of temozolomide (TMZ) and a therapeutically effective amount of noscapine or an analog thereof. In one aspect, the brain cancer is glioma. In another aspect, the glioma is glioblastoma multiforme.

Another embodiment of the present disclosure provides a method of treating a glioma patient, comprising, or alternatively consisting essentially of, or yet further consisting of, administering to the patient an effective amount of temozolomide (TMZ) or an analog thereof and an effective amount of noscapine.

A further embodiment of the present disclosure provides a method of treating a temozolomide (TMZ)-resistant brain cancer patient, comprising:

identifying a brain cancer patient that is resistant to TMZ; and

administering to the patient an effective amount of noscapine or an analog thereof.

In one aspect, the TMZ-resistant brain cancer patient is identified by a method comprising determining the activity or expression of the O-6-methylguanine-DNA methyltransferase (MGMT) gene in the patient. In one aspect, overexpression of MGMT identifies the brain cancer patient as resistant to TMZ. The expression level of a gene can be the protein expression level or mRNA expression level. In one aspect, the expression level is a mRNA expression level. Non-limiting examples of methods of determining intratumoral expression level of a gene include in situ hybridization, PCR, real-time PCR or microarray. In another aspect, the expression level is protein expression level. Non-limiting examples of methods of determining intratumoral protein expression level of a gene include immunohistochemistry, ELISA or protein microarrays.

In one aspect, the method further comprises, or alternatively consists essentially of, or yet further consists of, administering to the patient an effective amount of TMZ or an analog thereof.

A method of determining whether a brain cancer patient is likely suitable or not suitable for a therapy comprising the administration of noscapine or an analog thereof is also provided, comprising determining the activity or expression of the O-6-methylguanine-DNA methyltransferase (MGMT) gene in the patient, wherein overexpression of the MGMT gene identifies the patient as likely suitable for the therapy, or the lack of overexpression of the MGMT gene identifies the patient as likely not suitable for the therapy. In one aspect, the method further comprises administering to the patient that is likely suitable for the therapy a therapeutically effective amount of noscapine or an analog thereof.

Also provided in the disclosure is a pharmaceutical composition comprising an effective amount of temozolomide (TMZ) or an analog thereof, an effective amount of noscapine or an analog thereof, and a pharmaceutically acceptable carrier.

Kits or packages are also provided, comprising an effective amount of temozolomide (TMZ) or an analog thereof, and an effective amount of noscapine or an analog thereof. In one aspect, the kits or packages further comprise a primer, a probe, a microarray or an antibody or testing the activity or expression of the O-6-methylguanine-DNA methyltransferase (MGMT) gene.

### BRIEF DESCRIPTION OF THE DRAWING

FIG. 1 shows the in vivo effects of noscapine. TMZ-resistant U251 glioma cells (R) were implanted intracranially into nude mice. After 7 days, treatment was initiated. Mice were randomly divided into treatment groups: vehicle control (Con) (n=5); noscapine (Nos) (n=6); TMZ (n=4); and noscapine +TMZ (N+T) (n=4). The drugs were administered by gavage, at the following doses: noscapine at 225 mg/kg twice daily (6-8 hours apart); and TMZ at 5 mg/kg in a cycle of 7 days treatment followed by no TMZ treatment for 7 days. Survival was used as an endpoint; * signifies statistical significance (p<0.05).

### DETAILED DESCRIPTION

The present disclosure provides data to show that the nontoxic agent, noscapine, is cytotoxic to TMZ-resistant tumor cells. Noscapine caused reduced tumor growth and increased survival in animal models. Accordingly, the present disclosure demonstrates that noscapine and its analogs can be used as a therapeutic agent for the treatment of TMZ-resistant, recurrent brain tumors.

### DEFINITIONS

Unless defined otherwise, all technical and scientific terms used in this description have the same meaning as commonly understood by those skilled in the relevant art.

For convenience, the meaning of certain terms and phrases employed in the specification, examples, and appended claims are provided below. Other terms and phrases are defined throughout the specification.

The singular forms “a,” “an,” and “the” include plural reference unless the context clearly dictates otherwise.

“Cancer,” “neoplasm,” “tumor,” “malignancy” and “carcinoma,” used interchangeably herein, refer to cells or tissues that exhibit an aberrant growth phenotype characterized by a significant loss of control of cell proliferation. The methods and compositions of this invention particularly apply to malignant, pre-metastatic, metastatic, and non-metastatic cells.

“Drug” refers to any physiologically or pharmacologically active substance that produces a local or systemic effect in animals, particularly mammals and humans.

“Individual,” “subject,” “host,” and “patient,” terms used interchangeably in this description, refer to any mammalian subject for whom diagnosis, treatment, or therapy is desired. The individual, subject, host, or patient can be a human or a non-human animal. Thus, suitable subjects can include but are not limited to non-human primates, cattle, horses, dogs, cats, guinea pigs, rabbits, rats, and mice.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present invention for any particular subject depends upon a variety of factors including the activity of the specific compound employed, bioavailability of the compound, the route of administration, the age of the animal and its body weight, general health, sex, the diet of the animal, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. Studies in animal models generally may be used for guidance regarding effective dosages for treatment of diseases. In general, one will desire to administer an amount of the compound that is effective to achieve a scrum level commensurate with the concentrations found to be effective in vitro. Thus, where a compound is found to demonstrate in vitro activity, for example as noted in the Tables discussed below one can extrapolate to an effective dosage for administration in vivo. These considerations, as well as effective formulations and administration procedures are well known in the art and are described in standard textbooks. Consistent with this definition and as used herein, the term “therapeutically effective amount” is an amount sufficient to treat a specified disorder or disease or alternatively to obtain a pharmacological response treating a glioblastoma.

The term “administration” shall include without limitation, administration by ocular, oral, parenteral (e.g., intramuscular, intraperitoneal, inhalation, transdermal intravenous, ICV, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, ocular etc.) and can be formulated, alone or together, in suitable dosage unit formulations containing conventional non-toxic pharmaceutically acceptable carriers, adjuvants, excipients, and vehicles appropriate for each route of administration. The invention is not limited by the route of administration, the formulation or dosing schedule.

As used herein, “treating” or “treatment” of a disease in a patient refers to (1) preventing the symptoms or disease from occurring in an animal that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of this invention, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. In one aspect, the terms “treatment,” “treating,” “treat,” and the like refer to obtaining a desired pharmacological and/or physiologic effect in a brain tumor patient. The effect can be prophylactic in terms of completely or partially preventing brain tumor or symptom thereof and/or can be therapeutic in terms of a partial or complete stabilization or cure for brain tumor and/or adverse effect attributable to the brain tumor. Treatment covers any treatment of a brain tumor in a mammal, particularly a human. A desired effect, in particular, is reduction of tumor mass, inhibition of tumor mass increase, increased overall survival, increased progress free survival, reduced toxicity or reduced tumor recurrence.

### Treatment of Temozolomide-Resistant Brain Cancer

Noscapine has been extensively used as a cough suppressant for several decades, and known to cross the blood-brain-barrier. This drug is an orally bioavailable agent with an excellent tolerability profile; dose escalating studies have shown that noscapine has minimal toxicity and does not interfere with the immune response. Unlike other opium-derived products, noscapine does not have any analgesic, sedative, or euphoric properties and is not addictive.

The potential of noscapine to inhibit cancer cell growth in vitro was discovered a half century ago. The anti-tumor properties ascribed to noscapine have been primarily attributed to its ability to interfere with microtubule function, which leads to metaphase arrest of proliferating cells. Noscapine functions by binding to microtubules and disrupting the microtubule dynamics without causing mass accumulations of tubulin. Noscapine has been shown to have potent in vitro anti-tumor activity in a variety of tumor types. In vivo studies using the xenograft murine model for human non-small cell lung cancer, T cell lymphoma, prostate and breast cancer have demonstrated that noscapine has anti-cancer properties.

The present inventors have discovered, unexpectedly, that noscapine was effective in inhibiting glioma cell growth and treating temozolomide (TMZ)-resistant glioma.

Thus, one embodiment of the present disclosure provides a method of treating a temozolomide (TMZ)-resistant brain cancer patient, which comprises, or alternatively consists essentially of, or yet further consists of, administering to the patient an effective amount of noscapine or an analog thereof. In one aspect, the brain cancer is glioma. In another aspect, the glioma is glioblastoma multiforme. In one aspect, the method further comprises administering to the patient a therapeutically effective amount of TMZ. In a further aspect of this disclosure, the above noted methods further comprises, or alternatively consists essentially of, or yet further consists of, identifying a patient as having a TMZ-resistant brain cancer, and then after identifying the cancer as such, treating the patient in accordance with the disclosed methods.

A “temozolomide (TMZ)-resistant brain cancer patient,” as used herein, refers to a brain cancer patient that does not exhibit desired therapeutic benefit following administration of TMZ or an analog thereof. A desired therapeutic benefit, for instance, is amelioration of one or more brain cancer symptoms, partial or complete stabilization of tumor progression, reduction of tumor mass, inhibition of tumor mass increase, increased overall survival, increased progress free survival, reduced toxicity or reduced tumor recurrence.

“Temozolomide (TMZ),” also known as Temodar® and Temodal®, is an oral alkylating agent. TMZ is a derivative of imidazotetrazine, and is the prodrug of MTIC (3-methyl-(triazen-1-yl)imidazole-4-carboxamide). TMZ undergoes rapid chemical conversion in the systemic circulation at physiological pH to the active compound, MTIC (monomethyl triazeno imidazole carboxamide). A non-limiting example of a TMZ analog is MTIC. Other examples of TMZ analogs are disclosed in, e.g., U.S. Pat. No. 6,844,434 and U.S. Pat. No. 7,087,751.

“Noscapine,” also known as Narcotine, Nectodon, Nospen, Anarcotine and (archaic) Opiane, is a benzylisoquinoline alkaloid from plants of the Papaveraceae family. Noscapine was first isolated and characterized in chemical breakdown and properties in 1817 under the denomination of “Narcotine.” Methods of preparing noscapine are well known in the art. The IUPAC name of noscapine is (3S)-6,7-Dimethoxy-3-[(5R)-5,6,7,8-tetrahydro-4-methoxy-6-methyl-1,3-dioxolo (4,5-g)isoquinolin-5-yl]-1(3H)-isobenzofuranone and its structure is shown below.

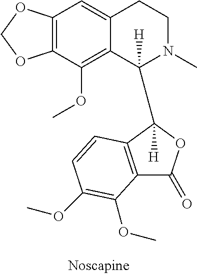

“Noscapine analogs,” including their metabolites, are known in the art. For instance, US 2010/0227878 provides Formula I and II, both of which showed anticancer activities. Methods of preparing and using these noscapine analogs are also provided in the patent application. U.S. Pat. No. 7,090,853, likewise, provides a range of noscapine analogs (termed “noscapine derivatives”). These noscapine analogs have also been shown to have anticancer activities. Other analogs are described in Anderson et al. (2005) J. Med. Chem. 48(23):7096-7098; Mishra et al. (2011) Biochm. Pharmacol. 82(2):110-121; and US Patent Publ. 2011/0286919A1.

Also provided, in one embodiment, is a method of inhibiting the growth of a brain cancer cell that is resistant to a temozolomide (TMZ) treatment, comprising contacting the brain cancer cell with an effective amount of noscapine or an analog thereof. The contacting can be in vivo or in vitro. Examples of noscapine analogs are provided above. In one aspect, the brain cancer is glioma. In one aspect, the glioma is glioblastoma multiforme. In one aspect, the method further comprises contacting the brain cancer cell with a therapeutically effective amount of TMZ. In a yet further aspect, the method is combined with a method to identify brain cancer cells as to whether or not the brain cancer cells are resistant to temozolomide (TMZ), prior to contacting the cells.

In some aspects, the method further comprises the administration or contacting of radiotherapy and/or another chemotherapy. Chemotherapies that can be used to treat brain tumor are known in the art. Selection of chemotherapeutic agents for a brain tumor patient depends on several factors, including the patient's age, Karnofsky Score and any previous therapy the patient has received. At www.neurooncology.ucla.edu/Performance/GlioblastomaMultiforme.aspx, the University of California at Los Angeles has published a list of anti-neoplastic agents that are suitable for treating brain tumors, which list is reproduced in Table 1 below.

TABLE 1
Known chemotherapeutic agents for treating brain tumors

| | | |
|---|---|---|
| 5FC | Accutane Hoffmann- | AEE788 Novartis |
| | La Roche | |
| AMG-102 | Anti Neoplaston | AQ4N (Banoxantrone) |
| AVANDIA | Avastin | BCNU |
| (Rosiglitazone | (Bevacizumab) | |
| Maleate) | Genetech | |
| BiCNU Carmustine | Carboplatin | CCI-779 |
| CCNU | CCNU Lomustine | Celecoxib (Systemic) |
| Chloroquine | Cilengitide (EMD | Cisplatin |
| | 121974) | |
| CPT-11 | Cytoxan | Dasatinib (BMS- |
| (CAMPTOSAR, | | 354825, Sprycel) |
| Irinotecan) | | |
| Dendritic Cell | Etoposide (Eposin, | GDC-0449 |
| Therapy | Etopophos, Vepesid) | |
| Gleevec (imatinib | GLIADEL Wafer | Hydroxychloroquine |
| mesylate) | | |
| Hydroxyurea | IL-13 | IMC-3G3 |
| Immune Therapy | Iressa (ZD-1839) | Lapatinib |
| | | (GW572016) |
| Methotrexate for | Novocure | OSI-774 |
| Cancer (Systemic) | | |
| PCV | Procarbazine | RAD001 Novartis |
| | | (mTOR inhibitor) |
| Rapamycin (Rapamune, | RMP-7 | RTA 744 |
| Sirolimus) | | |
| Simvastatin | Sirolimus | Sorafenib |
| SU-101 | SU5416 Sugen | Sulfasalazine |
| | | (Azulfidine) |
| Sutent (Pfizer) | Tamoxifen | TARCEVA (erlotinib |
| | | HCl) |
| Taxol | TEMODAR Schering- | TGF-B Anti-Sense |
| | Plough | |
| Thalomid (thalidomide) | Topotecan (Systemic) | VEGF Trap |
| VEGF-Trap | Vincristine | Vorinostat (SAHA) |
| XL 765 | XL 184 | XL765 |
| Zarnestra (tipifarnib) | ZOCOR (simvastatin) | |

### Personalized Treatment of Brain Cancer Patients

The present disclosure also provides personalized treatment methods for brain cancer patients. One benefit of personalized treatment is that patients that do not benefit from a treatment regimen do not need to be treated by that regimen and thus will not suffer the side effects brought about by the regimen. Conversely, patients that are identified to be able to benefit from a treatment regimen can receive the regimen at appropriate stage to maximize the benefit.

Accordingly, one embodiment of the present disclosure provides method of treating a temozolomide (TMZ)-resistant brain cancer patient, comprising:

identifying a brain cancer patient that is resistant to TMZ; and

administering to the patient an effective amount of noscapine or an analog thereof.

Methods of identifying a brain cancer patient resistant to TMZ treatment are known in the art. For instance, the overexpression of the O-6-methylguanine-DNA methyltransferase (MGMT) gene has been shown to lead to TMZ resistance. Thus, screening a sample from a brain cancer patient for the activity or expression of the MGMT gene can be used to identify whether the brain cancer patient will be resistant to TMZ.

In one aspect, overexpression of MGMT identifies the brain cancer patient as resistant to TMZ. In another aspect, the method further comprises administering to the patient an effective amount of TMZ or an analog thereof.

Treatment of Brain Cancer with Temozolomide and Noscapine

In one embodiment, the present disclosure provides a method of treating a primary central nervous system (CNS) tumor cancer patient (gliomas, menengiomas, pituitary adenomas), a CNS cancer cell metastasis from a systemic cancer, lung cancer cell, prostate cancer cell, breast cancer cell, hematopoietic cancer cell, ovarian cancer cell or brain cancer patient, whose tumor is resistant to temozolomide (TMZ), by administering to the patient an effective amount of temozolomide (TMZ) or an analog thereof, and a therapeutically effective amount of noscapine or an analog thereof. In one aspect, the brain cancer is glioma. In one aspect, the glioma is glioblastoma multiforme. Analogs of TMZ and noscapine are provided above.

Co-administration of these compositions can be administered concurrently or sequentially with other therapies such as radiation therapy, as known to those of skill in the art. The use of operative combinations is contemplated to provide therapeutic combinations that may lower total dosage of each component than may be required when each individual therapeutic method or composition is used alone. A reduction in adverse effects may also be noted. Thus, the present invention also includes methods involving co-administration of the compositions described herein with one or more additional active agents or methods. Indeed, it is a further aspect of this invention to provide methods for enhancing other therapies and/or pharmaceutical compositions by co-administering a composition of this invention. In co-administration procedures, the agents may be administered concurrently or sequentially. In one embodiment, the compounds described herein are administered prior to the other active agent(s), therapy or therapies. The pharmaceutical formulations and modes of administration may be any of those described herein or known to those of skill in the art.

### Compositions, Kits and Packages

Also provided is a pharmaceutical composition comprising an effective amount of temozolomide (TMZ) or an analog thereof and a therapeutically effective amount of noscapine or an analog thereof, effective to treating a temozolomide (TMZ)-resistant brain cancer patient or a central nervous system (CNS) tumor cancer patient (gliomas, menengiomas, pituitary adenomas), a CNS cancer cell metastasis from a systemic cancer, lung cancer cell, prostate cancer cell, breast cancer cell, hematopoietic cancer cell, ovarian cancer cell or brain cancer patient, whose tumor is resistant to temozolomide (TMZ). Analogs of TMZ and noscapine are provided above. In one aspect, the composition further comprises a pharmaceutically acceptable carrier.

It is contemplated that the TMZ or its analog, and the noscapine or its analog can be packaged separately to form a kit or package. Therefore, another embodiment of the present disclosure provides a kit or package comprising an effective amount of temozolomide (TMZ) or an analog thereof and a therapeutically effective amount of noscapine or an analog thereof.

In some aspect, the compositions, packages or kits further contain a primer, a probe, a microarray or an antibody or testing the activity or expression of the O-6-methylguanine-DNA methyltransferase (MGMT) gene.

### Formulations and Administration Routes and Schedules

The pharmaceutical compositions can be administered by any one of the following routes: ocular, oral, systemic (e.g., transdermal, intranasal or by suppository), or parenteral (e.g., intramuscular, intravenous or subcutaneous) administration. In some embodiments, the manner of administration is oral using a convenient daily dosage regimen that can be adjusted according to the degree of affliction. Compositions can take the form of tablets, pills, capsules, semisolids, powders, sustained release formulations, solutions, suspensions, elixirs, aerosols, or any other appropriate compositions. Another manner for administering compounds of described herein is inhalation.

The choice of formulation depends on various factors such as the mode of drug administration and bioavailability of the drug substance. For delivery via inhalation the compound can be formulated as liquid solution, suspensions, aerosol propellants or dry powder and loaded into a suitable dispenser for administration. There are several types of pharmaceutical inhalation devices-nebulizer inhalers, metered dose inhalers (MDI), mouth mask and dry powder inhalers (DPI). Nebulizer devices produce a stream of high velocity air that causes the therapeutic agents (which are formulated in a liquid form) to spray as a mist that is carried into the patient's respiratory tract. MDT's typically are formulation packaged with a compressed gas. Upon actuation, the device discharges a measured amount of therapeutic agent by compressed gas, thus affording a reliable method of administering a set amount of agent. DPI can dispense therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions can additional contain solid pharmaceutical excipients such as starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Liquid carriers, particularly for injectable solutions, include water, saline, aqueous dextrose, and glycols.

The amount of the composition in a formulation can vary within the full range employed by those skilled in the art. Typically, the formulation will contain, on a volume percent (v/v %) basis, from about 0.01-99.99 v/v % of a composition described herein based on the total formulation, with the balance being one or more suitable pharmaceutical excipients. In some embodiments, the composition is present at a level of about 1-80 v/v %.

Various delivery systems are known and can be used to administer a composition of the invention, e.g., intranasally or by inhalation, and the like. To determine patients that can be beneficially treated, a tissue sample can be removed from the patient and the cells are assayed for sensitivity to the agent.

Therapeutic amounts can be empirically determined and will vary with the pathology being treated, the subject being treated and the efficacy and toxicity of the composition as well as whether the composition is used alone or in combination with other agents of therapeutic methods. When delivered to an animal, the method is useful to further confirm efficacy of the agent.

Administration in vitro or in vivo can be effected in one dose, continuously or intermittently throughout the course of treatment and at various temperatures. Suitable temperature ranges include temperatures in the range of about 40° F. to about 120° F., or alternatively from about 50° F. to about 115° F., or alternatively from about 60° F. to about 100° F., or alternatively from about 65° F. to about 95° F., or alternatively from about 65° F. to about 115° F. or alternatively from about 65° F. to about 115° F. or alternatively from about 68° F. to about 110° F., or alternatively from about 68° F. to about 100° F., or alternatively from about 70° F. to about 95° F., or alternatively from about 72° F. to about 90° F., or alternatively from about 75° F. to about 85° F., or alternatively from about 75° F. to about 80° F., or alternatively at least 50° F., or alternatively from about 55° F., or alternatively at least 60° F., or alternatively at least 70° F., or alternatively from about 72° F., or alternatively at least 75° F., or alternatively at least 80° F., or alternatively at least 85° F., or alternatively at least 90° F., or alternatively at least 95° F., or alternatively at least 98° F., or alternatively at least 100° F., or alternatively at least 102° F., or alternatively at least 105° F.

Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician.

The pharmaceutical compositions can be administered orally, intranasally, ocularly, parenterally or by inhalation therapy, and may take the form of tablets, lozenges, granules, capsules, pills, ampoules, suppositories or aerosol form. They may also take the form of suspensions, solutions and emulsions of the active ingredient in aqueous or nonaqueous diluents, syrups, granulates or powders. In addition to an agent of the present invention, the pharmaceutical compositions can also contain other pharmaceutically active compounds or a plurality of compounds.

More particularly, the composition of the invention also referred to herein as the active ingredient, may be administered for therapy by any suitable route including oral, rectal, nasal, topical (including transdermal, aerosol, buccal and sublingual), vaginal, parenteral (including subcutaneous, intramuscular, intravenous and intradermal) and pulmonary. It will also be appreciated that the preferred route will vary with the condition and age of the recipient, and the disease being treated.

Ideally, the agent should be administered to achieve peak concentrations of the active composition at sites of disease. Desirable blood levels of the agent may be maintained by a continuous infusion to provide a therapeutic amount of the active ingredient within disease tissue or at the site of disease or tumor by multiple administrations.

Transdermal delivery systems manufactured as an adhesive disc or patch which slowly releases the compositions described herein for percutaneous absorption can be used. To this end, permeation enhancers can be used to facilitate transdermal penetration of the therapeutic agent. Suitable transdermal patches are described in, for example, U.S. Pat. No. 5,407,713; U.S. Pat. No. 5,352,456; U.S. Pat. No. 5,332,213; U.S. Pat. No. 5,336,168; U.S. Pat. No. 5,290,561; U.S. Pat. No. 5,254,346; U.S. Pat. No. 5,164,189; U.S. Pat. No. 5,163,899; U.S. Pat. No. 5,088,977; U.S. Pat. No. 5,087,240; U.S. Pat. No. 5,008,110; and U.S. Pat. No. 4,921,475.

### In Vitro Screens

This invention also provides screening assays to identify potential therapeutic agents of known and new compounds and combinations.

In one aspect, the assay requires contacting a first sample comprising suitable cells or tissue (“control sample”) with an effective amount of a composition of this invention and contacting a second sample of the suitable cells or tissue (“test sample”) with the agent to be assayed. The inhibition of growth of the first and second cell samples are determined. If the inhibition of growth of the second sample is substantially the same or greater than the first sample, then the agent is a potential drug for therapy. In one aspect, substantially the same or greater inhibition of growth of the cells is a difference of less than about 1%, or alternatively less than about 5% or alternatively less than about 10%, or alternatively greater than about 10%, or alternatively greater than about 20%, or alternatively greater than about 50%, or alternatively greater than about 90%. The contacting can be in vitro or in vivo. Means for determining the inhibition of growth of the cells are well know in the art and examples. In a further aspect, the test agent is contacted with a third sample of cells or tissue comprising normal counterpart cells or tissue to the control and test samples and selecting agents that treat the second sample of cells or tissue but does not adversely effect the third sample. For the purpose of the assays described herein, a suitable cell or tissue is one involved in hyperproliferative disorders such as cancer or other diseases as described herein. Examples of such include, but are not limited to cancer cell or tissue obtained by biopsy, blood, breast cells, colon cells, liver cells, or synovial fluid. In one aspect, the samples comprise a primary central nervous system (CNS) tumor cell (gliomas, menengiomas, pituitary adenomas), a CNS cancer cell metastasis from a systemic cancer, lung cancer cell, prostate cancer cell, breast cancer cell, hematopoietic cancer cell or ovarian cancer cell.

Efficacy of the test composition is determined using methods known in the art which include, but are not limited to cell viability assays or apoptosis evaluation.

The assays also are useful to predict whether a subject will be suitably treated by this invention by delivering a composition to a sample containing the cell to be treated and assaying for treatment which will vary with the pathology. In one aspect, the cell or tissue is obtained from the subject or patient by biopsy. Applicants provide kits for determining whether a pathological cell or a patient will be suitably treated by this therapy by providing at least one composition of this invention and instructions for use.

The test cells can be grown in small multi-well plates and is used to detect the biological activity of test compounds. For the purposes of this invention, the successful candidate drug will block the growth or kill the pathogen but leave the control cell type unharmed.

Compounds, agents and combinations thereof, identified by this method are further provided herein.

### Use of Compositions for Preparing Medicaments

The compositions of the present invention are also useful in the preparation of medicaments to treat a variety of brain cancers as described above. The methods and techniques for preparing medicaments of a composition are known in the art. For the purpose of illustration only, pharmaceutical formulations and routes of delivery are detailed herein.

Thus, one of skill in the art would readily appreciate that any one or more of the compositions described above, including the many specific embodiments, can be used by applying standard pharmaceutical manufacturing procedures to prepare medicaments to treat the many disorders described herein. Such medicaments can be delivered to the subject by using delivery methods known in the pharmaceutical arts.

### Pharmaceutical Delivery

Therapeutic amounts can be empirically determined and will vary with the pathology being treated, the subject being treated and the efficacy and toxicity of the composition as well as whether the composition is used alone or in combination with other agents of therapeutic methods. When delivered to an animal, the method is useful to further confirm efficacy of the agent.

Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician.

Suitable dosage formulations and methods of administering the agents can be readily determined by those of skill in the art. For example, TMZ can be administered at about 0.01 mg/kg to about 10 mg/kg, alternatively at about 0.1 mg/kg to about 5 mg/kg, or alternatively at about 0.5 mg/kg to about 3 mg/kg. Likewise, noscapine can be administered at about 1 mg/kg to about 500 mg/kg, alternatively at about 5 mg/kg to about 400 mg/kg, at about 10 mg/kg to about 300 mg/kg, at about 20 mg/kg to about 250 mg/kg, at about 30 mg/kg to about 200 mg/kg, or alternatively at about 50 mg/kg to about 200 mg/kg. When the compounds described herein are co-administered with another agent (e.g., as sensitizing agents) or therapy, the effective amount may be less than when the agent is used alone.

The pharmaceutical compositions can be administered orally, intranasally, ocularly, parenterally or by inhalation therapy, and may take the form of tablets, lozenges, granules, capsules, pills, ampoules, suppositories or aerosol form. They may also take the form of suspensions, solutions and emulsions of the active ingredient in aqueous or nonaqueous diluents, syrups, granulates or powders. In addition to an agent of the present invention, the pharmaceutical compositions can also contain other pharmaceutically active compounds or a plurality of compounds.

More particularly, the composition of the invention also referred to herein as the active ingredient, may be administered for therapy by any suitable route including oral, rectal, nasal, topical (including transdermal, aerosol, buccal and sublingual), vaginal, parenteral (including subcutaneous, intramuscular, intravenous and intradermal) and pulmonary. It will also be appreciated that the preferred route will vary with the condition and age of the recipient, and the disease being treated.

Ideally, the agent should be administered to achieve peak concentrations of the active composition at sites of disease. Desirable blood levels of the agent may be maintained by a continuous infusion to provide a therapeutic amount of the active ingredient within disease tissue or at the site of disease or tumor by multiple administrations.

The following examples are intended to illustrate, but not limit, the invention.

### Example 1

This example demonstrates the antitumor effectiveness of noscapine with brain cancer cells resistant to temozolomide (TMZ), in vitro and in vivo.

Using 4 different TMZ-resistant glioma cell lines, this example generated data that noscapine caused cell death of these cells. More specifically, noscapine caused an arrest in the G2/M phase of cell cycle and prolonged arrest causes cell cytotoxicity. It was also demonstrated that noscapine decreased the migration and invasion of TMZ-resistant tumor cells.

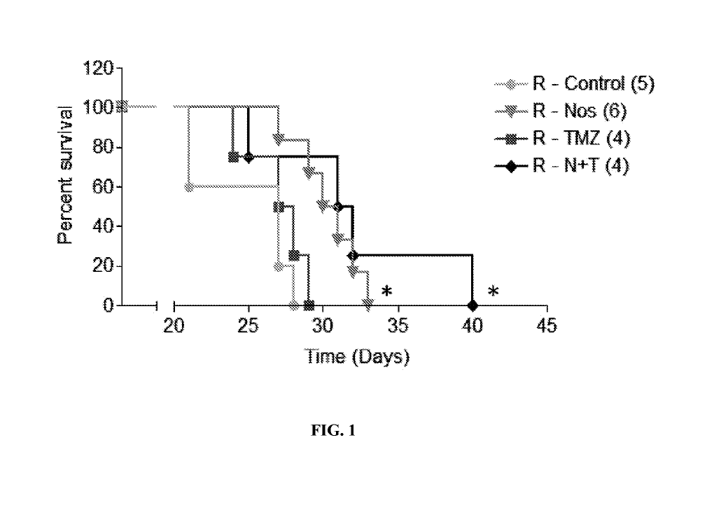

Furthermore, in the intracranial rodent xenograft tumor model, noscapine significantly enhanced survival of TMZ-resistant tumor bearing animals. Thus noscapine demonstrated potent anti-tumor properties towards TMZ-resistant brain tumors. As shown in FIG. 1, TMZ-resistant U251 glioma cells (R) were implanted intracranially into nude mice. After 7 days, treatment was initiated. The drugs were administered by gavage, at the following doses: noscapine at 225 mg/kg twice daily (6-8 hours apart); and TMZ at 5 mg/kg in a cycle of 7 days treatment followed by no TMZ treatment for 7 days.

FIG. 1 shows that animals treated with both TMZ and noscapine had significantly higher survival rate, in particularly after 33 days.

Unless otherwise defined, all technical and scientific terms used herein have the same meaning as commonly understood by one of ordinary skill in the art to which this invention belongs. All nucleotide sequences provided herein are presented in the 5′ to 3′ direction.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

1. A method of treating a temozolomide (TMZ)-resistant brain cancer patient, comprising administering to the patient an effective amount of noscapine or an analog thereof.

2. The method of claim 1, wherein the brain cancer is glioma, or glioblastoma multiforme.

3. The method of claim 1, further comprising administering to the patient an effective amount of TMZ or an analog thereof.

4. The method of claim 1, further comprising administering to the patient a chemotherapy or radiotherapy.

5. A method of treating a temozolomide (TMZ)-resistant glioma patient, comprising administering to the patient an effective amount of noscapine and a therapeutically effective amount of TMZ.

6. A method of inhibiting the growth of a brain cancer cell that is resistant to a temozolomide (TMZ) treatment, comprising contacting the brain cancer cell with an effective amount of noscapine or an analog thereof.

7. The method of claim 6, wherein the contacting is in vivo, or in vitro.

8. A method of treating a brain cancer patient, comprising administering to the patient an effective amount of temozolomide (TMZ) and a therapeutically effective amount of noscapine or an analog thereof.

9. The method of claim 8, wherein the brain cancer is glioma, or glioblastoma multiforme.

10. A method of treating a glioma patient, comprising administering to the patient an effective amount of temozolomide (TMZ) or an analog thereof and an effective amount of noscapine.

11. A method of treating a temozolomide (TMZ)-resistant brain cancer patient, comprising administering to the patient identified as having a tumor that is resistant to TMZ an effective amount of noscapine or an analog thereof.

12. The method of claim 11, wherein the TMZ-resistant brain cancer patient is identified by a method comprising determining the activity or expression of the O-6-methylguanine-DNA methyltransferase (MGMT) gene in the patient, and wherein overexpression of MGMT identifies the brain cancer patient as resistant to TMZ.

13. The method of claim 11, further comprising administering to the patient an effective amount of TMZ or an analog thereof, wherein the TMZ is administered prior to, concurrently or subsequent to noscapine or an analog thereof.

14. A method of determining whether a brain cancer patient is likely suitable or not suitable for a therapy comprising the administration of noscapine or an analog thereof, comprising determining the activity or expression of the O-6-methylguanine-DNA methyltransferase (MGMT) gene in the patient, wherein overexpression of the MGMT gene identifies the patient as likely suitable for the therapy, or the lack of overexpression of the MGMT gene identifies the patient as likely not suitable for the therapy.

15. The method of claim 14, further comprising administering to the patient that is likely suitable for the therapy a therapeutically effective amount of noscapine or an analog thereof.

16. A pharmaceutical composition comprising an effective amount of temozolomide (TMZ) or an analog thereof suitable to treat a temozolomide (TMZ)-resistant brain cancer patient or an amount effective to treat a central nervous system (CNS) tumor cancer patient (gliomas, menengiomas, pituitary adenomas), a CNS cancer cell metastasis from a systemic cancer, lung cancer cell, prostate cancer cell, breast cancer cell, hematopoietic cancer cell, ovarian cancer cell or brain cancer patient, whose tumor is resistant to temozolomide (TMZ), and a pharmaceutically acceptable carrier.

17. A kit or package comprising an effective amount of temozolomide (TMZ) or an analog thereof, and an effective amount of noscapine or an analog thereof, and optionally a primer, a probe, a microarray or an antibody or testing the activity or expression of the O-6-methylguanine-DNA methyltransferase (MGMT) gene.
